(19) United States
(12) Patent Application Publication
(10) Pub. No.: US 2017/0166978 A1
(43) Pub. Date: Jun. 15, 2017

(54) METHODS FOR TREATING CANCER

(71) Applicant: Wayne Holman, Miami, FL (US)

(72) Inventor: Wayne Holman, Miami, FL (US)

(21) Appl. No.: 15/378,883

(22) Filed: Dec. 14, 2016

Related U.S. Application Data
(60) Provisional application No. 62/267,806, filed on Dec. 15, 2015.

(51) Int. Cl. C12Q 1/68; G01N 33/50; A61K 39/00

(52) U.S. Cl. CPC C12Q 1/6886; A61K 39/0011; G01N 33/505; G01N 33/5011; A61K 2039/53; A61K 48/00

(57) ABSTRACT

A method for treating cancer comprising the steps of genetically sequencing a patient healthy tissue, genetically sequencing a patient tumor tissue, comparing the genetic sequences of the healthy tissue and tumor tissue to identify one or more mutations specific to the tumor tissue, generating a library of 9-mers having one or more peptide fragments specific to the tumor tissue, and identifying a 9-mer that elicits the strongest immune response in the patient.

This application claims the benefit of U.S. Provisional Patent Application No. 62/267,806 filed on Dec. 15, 2015, which is hereby incorporated by reference in its entirety as if set forth herein.

### BACKGROUND OF THE INVENTION

Field of the Invention

The present invention is directed to methods for treating cancer. More specifically, the present invention is directed to methods for utilizing the patient's own immune system to treat and potentially cure cancer in humans.

Background

The present invention relates to methods that use the patient's own immune system to treat cancer in humans by generating cytotoxic T cells or T cells having a high affinity to neo-antigens present on a malignant tumor. Such cytotoxic T cells are part of the human adaptive immune system, and will attack and destroy cells present in the human body that express mutated “non-self” proteins.

Much of current research into T cell therapy for cancer relates to generating an endogenous (naturally occurring inside the human host) T cell response against shared self-proteins present in the cancer of many individuals. However, the immune system is designed to avoid the generation of endogenous high affinity T cells against the self-protein target. Other methods employ synthetic T cells with genetic modifications to the T cell receptor that create a high affinity to a specific self-protein associated with the tumor. This method may increase the efficacy of the treatment, but has potential safety issues. Previous administrations of synthetic genetically modified T cells have led to patient deaths due to both “off-target” effects where the attacking T cells recognize and attack a different peptide than they are designed for, and “on-target” effects where the T cells recognize and attack the same peptide they are designed for, but on non-tumor tissue of the patient. The genetically modified T cell receptor (TCR) has never been in the patient who is receiving the modified T cells, and predicting what tissues those T cells will attack is an imprecise science.

It has been postulated that certain proteins referred to as neo-antigens or super-antigens may exist that are non-self and thus capable of generating a clone of endogenous high affinity T cells in a human cancer patient. Such neo-antigens are believed to be tumor-specific antigens derived from mutated proteins that are present only in the tumor, and not in any other patient self-tissue. Thus the expression of these proteins is likely limited such that they occur only in cancers that cause evident human disease. If the expression of the neo-antigen is high enough it would likely lead to destruction of the tumor by development of an endogenous T cell clone before the tumor grew into a diagnosable cancer. The present invention aims to increase the expression level of the neo-antigen by the tumor cells. A greater expression of the neo-antigen by the tumor cells will amplify the signal to the existing high affinity T cell clone and stimulate the destruction of the tumor by the immune system.

In some situations, however, the expression of the neo-antigen may be high, the size and activity of the endogenous T cell clone against that neo-antigen may be high, but the tumor may elaborate proteins that down-regulate the immune attack. Several drugs have been developed that inhibit the ability of the tumor to down-regulate such a T cell driven immune attack on tumors including drugs using antibodies against CTLA4, PD-1 and PDL-1. Some of the proteins the tumor produces to down-regulate the immune attack are called checkpoints. The drugs that inhibit those checkpoints are called checkpoint inhibitors (CPIs).

Research has shown that the patients who respond best to CPIs have the highest mutational load and are likely to have neo-antigens present in the tumor. The higher the mutational load the greater the number of mutations present in the tumor. The greater the number of mutations in the tumor, the greater the likelihood that one of those mutations looks like a non-self antigen or neo-antigens, and thus is capable of generating a high affinity endogenous T cell clone.

The present invention overcomes present treatment limitations by inducing a high affinity T cell attack on a cancerous tumor, either alone or in combination with checkpoint inhibitors and without introducing either exogenous T cells or synthetic genetically modified T cells into the patient.

### SUMMARY OF THE INVENTION

A method for treating cancer comprising the steps of genetically sequencing a patient healthy tissue, genetically sequencing a patient tumor tissue, comparing the genetic sequences of the healthy tissue and tumor tissue to identify one or more mutations specific to the tumor tissue, generating a library of 9-mers having one or more peptide fragments specific to the tumor tissue, and identifying a 9-mer that elicits the strongest immune response in the patient. A method for treating cancer further comprising the step of inducing a tumor in the patient to increase production of the identified 9-mer. A method for treating cancer further comprising the step of inducing a tumor in the patient to increase production of a full length protein associated with the identified 9-mer. A method for treating cancer further comprising the step of inducing a tumor in the patient to increase production of mutated proteins. A method for treating cancer further comprising the step of inducing a tumor in the patient to increase production of immune modulators. A method for treating cancer further comprising the step of administering one or more immune modulators to the patient. A method for treating cancer further comprising the step of administering one or more mRNA vaccines to the patient. A method for treating cancer further comprising the step of administering one or more immune modulators and one or more mRNA vaccines to the patient.

### BRIEF DESCRIPTION OF THE DRAWINGS

FIG. 1 is a flow chart identifying the steps of an embodiment of the present invention.

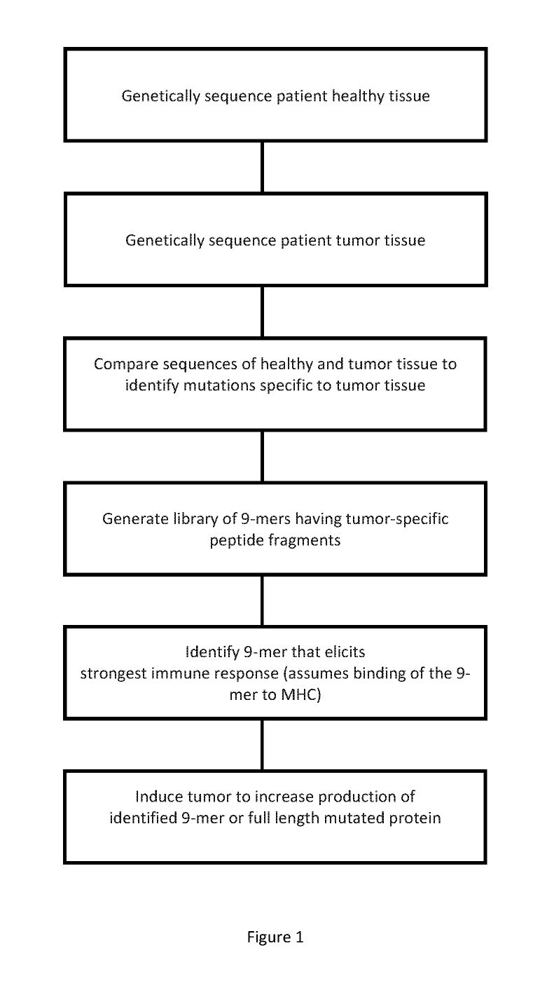

FIG. 2 is a flow chart identifying the steps of an embodiment of the present invention.

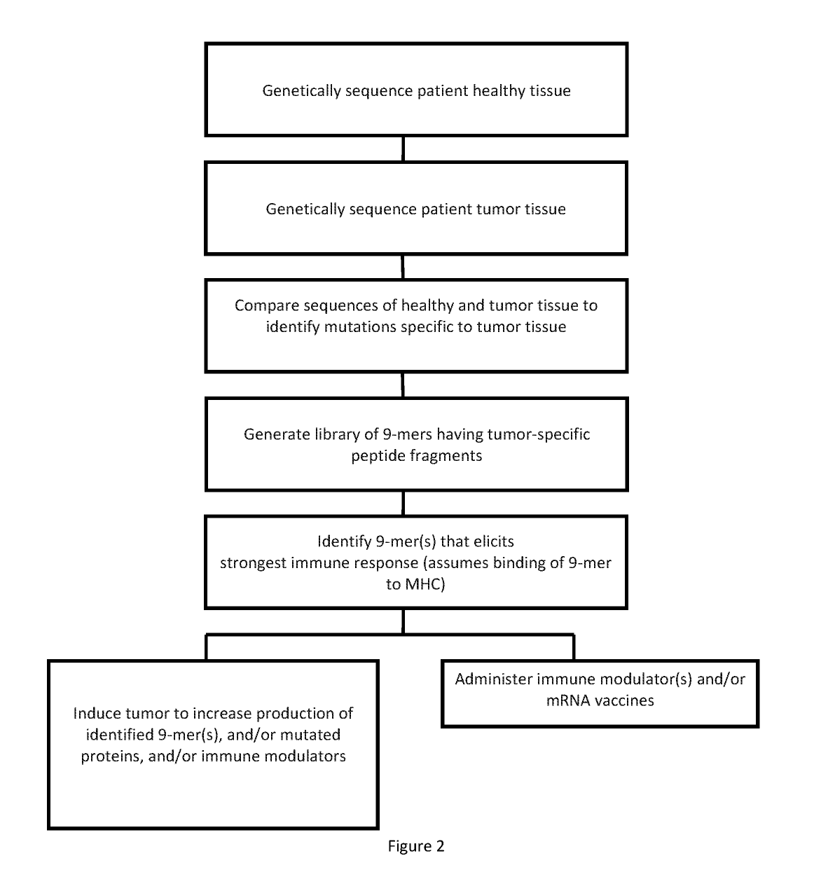

### DESCRIPTION OF THE PREFERRED EMBODIMENTS

The following description is presented to enable any person skilled in the art to practice the present invention. For purposes of explanation, specific nomenclature is set forth to provide a thorough understanding of the present invention. Descriptions of specific embodiments or applications are provided only as examples. Various modifications to the embodiments will be readily apparent to those skilled in the art, and general principles defined herein may be applied to other embodiments and applications without departing from the spirit and scope of the invention. Thus, the present invention is not intended to be limited to the embodiments shown, but is to be accorded the widest possible scope consistent with the principles and features disclosed herein.

The present invention relates to a method that uses the immune system to treat cancer in humans by generating cytotoxic T cells having a high affinity to neo-antigens present on and in a malignant tumor. Such cytotoxic T cells are part of the human adaptive immune system, and will attack and destroy cells present in the human body that express “non-self” proteins.

T cells are activated by foreign antigens present on the surface of antigen-presenting cells. T cells recognize fragments of protein antigen that have been partly degraded into peptide fragments inside the antigen-presenting cell. The peptide fragments are then carried to the surface of the antigen-presenting cell on special molecules called major histocompatibility complex or “MEW” proteins.

T cells are characterized by the presence of a T cell receptor (TCR) on the cell surface. The TCR of a T cell is specific to a particular antigen. T cells are activated by the presence of the specific peptide fragments of protein antigen complexed with MEW proteins on the surface of the antigen-presenting cell matching their TCR. TCRs recognize the peptide fragment bound to the MHC protein. They do not recognize whole proteins. When a T cell encounters a peptide or peptide fragment in the context of an MHC protein on the outside of another cell that matches the specificity of its TCR, the T cell will replicate and create effector T cells with the same specificity.

Those effector T cells will then attack and destroy cells expressing that protein or peptide. Endogenous T cells will not generate high affinity TCRs with specificities for a self-protein or peptide. Using next generation genetic sequencing (NGS), both a patient's healthy tissue and tumor tissue can be sequenced and compared to identify mutations specific to the tumor. Those genetic mutations specific to the patient's tumor can then be used to create a library of peptides likely to be complexed with MHC proteins on the surface of tumor cells.

In one embodiment of the present invention, sequences for a patient's healthy tissue and tumor issue are compared to identify mutations specific to the tumor. A library of peptides having nine amino acids (9-mers) is then created that covers some or all of the tumor-specific peptide fragments. The neo-antigen may be considered to be the mutated protein or the epitope—the specific part of the mutated protein—that elicits the immune response. Since the 9-mer is the key piece that is responsible for the immune response, the 9-mer may be considered to be the antigen, or the antigenic part of the mutated protein or the epitope. In various exemplary embodiments, these are generally 9-mers, but occasionally slightly longer or shorter peptides may be complexed with and presented by MHC proteins. For purposes of this description the discussion will refer to 9-mers, but it will be understood by those of skill in the art that the present invention is not limited to the use of 9-mers and that longer or shorter peptides may be used.

From this library of 9-mers, there are alternative ways to identify and select the best peptide 9-mers from the library of tumor-specific peptide fragments that elicit the strongest immune response. The library of peptide 9-mers can be presented to the host's naturally occurring endogenous T cells in the context of MHC proteins on antigen presenting cells of the patient. In one embodiment of the present invention, each of peptide 9-mers in the library may be exposed to the patient's T cells ex vivo (outside of the body) to identify which 9-mer(s) generate the highest T cell response as measured by cytokine production. In various alternative embodiments, the 9-mers can be selected based on other types of ex vivo testing or by the use of predictive algorithms.

Once the tumor-specific peptide 9-mers or neo-antigens expected to generate the highest T cell response have been identified and selected, a delivery platform is developed to induce the patient's tumor to produce the mutated protein or the 9-mer in increased quantities. In one embodiment, a DNA plasmid of the peptide 9-mer and/or the full-length protein from which it is derived is constructed. The DNA plasmid may then be administered, such as by direct injection into one or several of the tumors in the patient. DNA plasmids covering one or more 9-mers, other portions of mutated proteins, or entire mutated proteins may be used. Electroporation, the administration of an electric current to foster uptake of the DNA plasmid by the tumor cells, may also be used to improve the 9-mer infusion process.

Other techniques for improving the infusion process may be used. For example, it is possible to generate lipid nanoparticles that may preferentially deliver the plasmid to the tumor cells. At present, this technique would be more difficult and require different delivery vehicles for each tumor. It is also possible to inject the protein itself into the tumor site and hope it is taken up by anaphase-promoting complex (APC).

To augment the immune response against the tumor further, in various exemplary embodiments an mRNA of the peptide 9-mer and/or the full-length protein from which it is derived may also be constructed. This mRNA may be administered to the same patient at or around the same time as the DNA plasmid via subcutaneous, intramuscular, or intra-dermal injection into a non-tumor site with or without electroporation. This is basically combining two methods that can be employed to generate an immune attack on the tumor that expresses a particular neo-antigen. For example, in an exemplary embodiment the step of electroporation of DNA for the neo-antigen or mutated protein directly into the tumor cells may be combined with injection of mRNA for the same neo-antigen or 9-mer into the patient at a non-tumor site. The mRNA injection if administered would serve to allow greater presentation of the antigen to dendritic cells, macrophages and natural killer cells. Such cells would then traffic to the lymph nodes where they would present the neo-antigen MHC complex to circulating and resident T cells to increase the endogenous T cell specific immune response to the tumor.

Once the tumor has been infused with the selected DNA plasmid, the tumor itself produces larger amounts of the neo-antigen. The neo-antigen will be processed and the 9-mer will be on the outside of the tumor cell and on antigen presenting cells (APCs) such as dendritic cells, macrophages, and natural killer cells in the tumor microenvironment. This presentation of the neo-antigen will cause the activation of and an increase in numbers of the antigen specific T cell clones for the neo-antigen that is now being expressed in greater amounts by the tumor. It may also induce activation and an increase in the number of other T cells via epitope spreading.

The infusion and electroporation of the tumor tissue with the selected DNA plasmid encoding the neo-antigen protein or peptide 9-mer may also induce the tumor to activate defenses that inhibit T cell response. In various embodiments of the present invention immune modulators, including but not limited to checkpoint inhibitors (CPIs), immune activators, and cytokines, may be administered as part of the treatment method to counter such defenses. The immune modulators will simultaneously prevent the tumor from inhibiting the T cell response and the innate immune response, and also potentially potentiate or enhance the immune response. In one embodiment of the present invention, an infusion of an immune modulator such as a CPI, including but not limited to an antibody against the following targets PD-1, PDL-1, LAG-3, and TIM-3, or an immune activator including but not limited to antibodies against the following targets GITR, CD40, CD-27, OX40 may be used. Such CPIs or drugs can be combined with the DNA plasmid encoding the neo-antigen 9mer in any suitable way, including without limitation with the CPIs or drugs being given as proteins via IV infusion, subcutaneous injection, or as DNA plasmids via direct injection and electroporation at the tumor site.)

The DNA plasmid for the 9-mer(s) and/or full length mutated proteins may also be administered in combination with one or more other DNA plasmids encoding for other therapeutic proteins, such as CPIs, immune activators, cytokines, immune modulators. The DNA plasmid can encode the neo-antigen protein alone or it can also encode other proteins sought to be produced by the tumor cells, such as CPIs, cytokines, immune activators or other proteins that will overcome inhibitors of the local immune attack on the tumor, or proteins that will augment the immune attack on the tumor. In other words one DNA plasmid can be injected and electroporated into the tumor that encodes both the neo-antigen (s) and the immune modulating drugs, or multiple discrete DNA plasmids can be infused encoding for various elements of the therapeutic cocktail.

Inducing production of the 9-mers, mutated proteins, and delivery of immune modulators by infusion directly to the tumor and avoiding the systemic administration of T cells has a variety of potential benefits including reducing system toxicities, allowing for administration of otherwise intolerable drugs, concentrating effects at the tumor site, providing for high affinity T cell expansion of endogenous T cells and avoiding potentially harmful or fatal toxicity from synthetic TCRs, providing greater efficacy against self-antigens than low affinity T cells, avoiding difficult exogenous T cell expansion, and allowing for the potential cure by efficiently targeting patient-specific and relatively unique neo-antigens. It also has a variety of potential benefits as compared to cancer vaccine approaches, including the targeting neo-antigens, and the eliminating of the need for trafficking of subsequently activated T cells to the tumor.

What is claimed is:

1. A method for treating cancer, comprising the steps of:
genetically sequencing a patient healthy tissue;
genetically sequencing a patient tumor tissue;
comparing the genetic sequences of the healthy tissue and tumor tissue to identify one or more mutations specific to the tumor tissue;
generating a library of 9-mers having one or more peptide fragments specific to the tumor tissue; and
identifying a 9-mer that elicits the strongest immune response in the patient;

2. The method of claim 1, further comprising the step of inducing a tumor in the patient to increase production of the identified 9-mer.

3. The method of claim 1, further comprising the step of inducing a tumor in the patient to increase production of a full length protein associated with the identified 9-mer.

4. The method of claim 1, further comprising the step of inducing a tumor in the patient to increase production of mutated proteins.

5. The method of claim 1, further comprising the step of inducing a tumor in the patient to increase production of immune modulators.

6. The method of claim 1, further comprising the step of administering one or more immune modulators to the patient.

7. The method of claim 1, further comprising the step of administering one or more mRNA vaccines to the patient.

8. The method of claim 1, further comprising the step of administering one or more immune modulators and one or more mRNA vaccines to the patient.
